(19) United States
(12) Patent Application Publication
(10) Pub. No.: US 2018/0140540 A1
(43) Pub. Date: May 24, 2018

(54) TOPICAL CLEANSING COMPOSITION WITH PREBIOTIC/PROBIOTIC ADDITIVE

(71) Applicant: GOJO Industries, Inc., Akron, OH (US)

(72) Inventors: Sarah Gantz, Fairlawn, OH (US); Amanda Copeland, Seville, OH (US); Carrie Anne Zapka, Austintown, OH (US)

(21) Appl. No.: 15/819,694

(22) Filed: Nov. 21, 2017

Related U.S. Application Data
(60) Provisional application No. 62/425,677, filed on Nov. 23, 2016.

(51) Int. Cl. A61K 8/99; A61Q 19/10; A61Q 17/00

(52) U.S. Cl. CPC A61K 8/99; A61K 2800/85; A61Q 17/005; A61Q 19/10

(57) ABSTRACT

A topical cleansing composition for restoring skin's natural balance of bacteria and/or increasing the production and/or activity of antimicrobial peptides is provided. The topical cleansing composition includes about 0.005 wt. % to 15.0 wt. % of an active ingredient that is one or more of a probiotic, probiotic derivative, prebiotic, and at least one primary and at least one secondary surfactant.

### RELATED APPLICATIONS

This application claims priority to and the benefit of U.S. Provisional Patent Application Ser. No. 62/425,677, entitled “TOPICAL CLEANSING COMPOSITION WITH PREBIOTIC/PROBIOTIC ADDITIVE” and filed Nov. 23, 2016, the entire disclosure of which is incorporated herein by reference.

### BACKGROUND

The skin is the human body's largest organ, colonized by a diverse range of microorganisms, the majority of which are harmless or even beneficial to their host. These microorganisms often provide vital functions that the human genome has not yet evolved to perform. In this way, the skin constantly regulates a balance between host-human and microorganism. Disruptions in this delicate balance, on either side, can result in serious skin disorders or infections.

Pathogens on the skin are known to cause illness and may be easily transmitted from one person to another. Some pathogens stick strongly to skin. Typically, when pathogens stick to skin, they are more difficult to remove or kill using traditional approaches to skin cleaning and disinfection such as washing with a conventional soap or waterless sanitizer. Pathogens that are stuck to skin are more dangerous because they remain on the skin longer. The longer the pathogen is on the skin, the more the chance that they will either cause infections on the person with them or be shared with other people.

The overuse of antibiotics is contributing an increase in the types and numbers of antibiotic-resistant pathogens, and infections from these pathogens are becoming more dangerous. There is an increasing interest in finding alternative ways to control pathogens without the use of more antimicrobials. Probiotics are being used to control microbes on skin in new ways that do not require the use of antimicrobials. Probiotics are live or inactivated microorganisms that, when either present as part of the normal microbiota or when administered in adequate amounts, confer a health or cosmetic benefit on the host. Benefits from probiotics can be from the microbial components directly or can come from the byproducts of bacterial growth.

Antimicrobial peptides (AMPs) comprise a wide range of natural and synthetic peptides that are made of oligopeptides containing a varying number of amino acids. AMPs may be produced by a host, or by the skin microbiota itself. AMPs are essential components of host defense against infections present in all domains of life. AMPs are produced by all complex organisms and have diverse and intricate antimicrobial activities. As a whole, these peptides demonstrate a broad range of antiviral and antibacterial activities through an array of modes of action. AMPs have been found to kill Gram-negative and Gram-positive bacteria, certain viruses, parasites and fungi. Some research suggests that they can also enhance the internal immunity of complex organisms against a broad range of bacteria and viruses. In addition to the innate immune system present in all animals, vertebrates evolved an adaptive immune system based on specific recognition of antigens. Increasing evidence suggests that AMPs released in response to an invasion of microbial can activate adaptive immunity by attracting antigen-presenting dendritic cells to the invasion site.

Therefore, it would be beneficial to design a new cleansing composition that is safe for topical use, restores the natural balance of bacteria on the skin, including decreasing the adherence of pathogens on the skin, and can also increase the production and/or activity of antimicrobial peptides.

### SUMMARY

According to some exemplary embodiments, a topical cleansing composition for restoring skin's natural balance of bacteria is provided. The topical composition includes about 0.005 wt. % to 15.0 wt. % of an active ingredient that is one or more of a probiotic, a probiotic derivative, and a prebiotic. The topical composition also includes at least one primary and at least one secondary surfactant. Application of the topical cleansing composition reduces pathogen binding on the surface of the skin by an amount that is statistically significant compared to an otherwise identical topical composition without the active ingredient.

In some exemplary embodiments, the primary surfactant is sodium laureth sulfate and the secondary surfactant is selected from one or more of cocamidopropyl betaine, disodium cocoamphodiacetate, cocamiopropyl hydroxysultaine, and lauryl glucoside.

In some exemplary embodiments, the active ingredient is a probiotic or probiotic derived ingredient, which can be selected from a strain of one or more the following: Lactobacillus, strains and derivatives of Clostridia, strains and derivatives of Bifidobacterium, strains and derivatives of Saccharomyces, strains and derivatives of Lactococcus, strains and derivatives of Pedicoccus, strains and derivatives of Enterococcus, strains and derivatives of Escherichia, strains and derivatives of Alcaligenes, strains and derivatives of Corynebacterium, strains and derivatives of Bacillus, and strains and derivatives of Propionibacterium. In some exemplary embodiments, the probiotic or probiotic derived ingredient is a Bacillus ferment.

In some exemplary embodiments, the topical cleansing composition comprises from about 0.05 to about 5.0 wt. % or from about 0.1 to about 1.0 wt. % of the active ingredient, based on the total weight of the topical cleansing composition.

In some exemplary embodiments, the topical cleansing composition contains up to about 20.0 wt. % of a humectant as the skin conditioning agent, selected from the group consisting of propylene glycol, hexylene glycol, 1,4-dihydroxyhexane, 1,2,6-hexanetriol, sorbitol, butylene glycol, caprylyl glycol, propanediols, such as methyl propane diol, dipropylene glycol, triethylene glycol, glycerin (glycerol), polyethylene glycols, ethoxydiglycol, polyethylene sorbitol, glyceryl caprylate/caprate, and combinations thereof.

In some exemplary embodiments, the topical cleansing composition also contains up to about 20.0 wt. % of one or more plug preventing additives, based on the total weight of the topical cleansing composition.

In some exemplary embodiments, the topical cleansing composition also contains up to 10.0 wt. % of a moisturizing ester, selected from the group consisting of selected from the group consisting of cetyl myristate, cetyl myristoleate, and other cetyl esters, diisopropyl sebacate, isopropyl myristate, and combinations thereof.

In some exemplary embodiments, the topical cleansing composition further comprises a carrier, which can be water.

Further exemplary embodiments relate to a method of skin treatment for reducing skin irritation. The method includes applying a topical cleansing composition to a skin surface, wherein the topical composition comprises about 0.005 wt. % to about 15.0 wt. % of an active ingredient and at least one primary and at least one secondary surfactant. The active ingredient comprises one or more of a probiotic, a probiotic derivative, and prebiotic. The method further includes rinsing the topical cleansing composition off with water. The topical composition reduces IL-8 concentration by a statistically significant amount, as compared to an otherwise identical topical composition without the active ingredient.

In some exemplary embodiments, the topical cleansing composition decreases the concentration of IL-8 by at least about 78%, relative to an otherwise identical topical composition without the active ingredient.

Further exemplary embodiments relate to a topical cleansing composition for stimulating the production of antimicrobial peptides on the skin. The topical cleansing composition comprises about 0.005 wt. % to about 15.0 wt. % of an active ingredient and at least one primary and at least one secondary surfactant. The active ingredient comprises one or more of a probiotic, a probiotic derivative, and prebiotic. The topical cleansing composition increases the concentration of antimicrobial peptides on skin by a statistically significant amount, as compared to an otherwise identical topical composition without the active ingredient.

In some exemplary embodiments the topical cleansing composition increases the production and/or activity of defensins by at least about 44% and the production and/or activity of cadherins by at least 57%, both relative to an otherwise identical topical composition without the active ingredient.

### BRIEF DESCRIPTION OF THE FIGURES

FIG. 1 illustrates an exemplary graph of the relative Interleukin 8 expression in topical compositions containing 1.0 wt. % Bonicel™ compared to a control.

FIG. 2 illustrates an exemplary graph of the Involiucrin expression in compositions containing 1.0 wt. % Bonicel™ compared to a control.

FIG. 3 illustrates an exemplary graph of the DSC3 expression in compositions containing 0.1 wt. % Bonicel™ compared to a control.

FIG. 4 illustrates an exemplary graph of the HBD-2 expression in compositions containing 0.1 wt. % Bonicel™ and 1.0 wt. % Bonicel™ compared to a control.

FIG. 5 illustrates an exemplary graph of the response of Staphylococcus aureus adhesion and invasion potential when treated with a probiotic Bacillus ferment.

### DETAILED DESCRIPTION

Unless otherwise defined, all technical and scientific terms used herein have the same meaning as commonly understood by one of ordinary skill in the art to which this application pertains. Although other methods and materials similar or equivalent to those described herein may be used in the practice or testing of the exemplary embodiments, exemplary suitable methods and materials are described below. In case of conflict, the present specification including definitions will control. In addition, the materials, methods, and examples are illustrative only and not intended to be limiting of the general inventive concepts.

The terminology as set forth herein is for description of the exemplary embodiments only and should not be construed as limiting the application as a whole. Unless otherwise specified, “a,” “an,” “the,” and “at least one” are used interchangeably. Furthermore, as used in the description of the application and the appended claims, the singular forms “a,” “an,” and “the” are inclusive of their plural forms, unless contradicted by the context surrounding such.

The term “microorganism” or “microbe” as used herein, refers to a tiny organism, such as a virus, protozoan, fungus, or bacterium that can only be seen under a microscope. The collection of microorganisms that live in an environment makes up a microbiota. For example human skin microbiota is all of the microbes on skin or a hospital microbiota would include all of the microbes in a hospital building. The term microbiome is used when referring to the entire habitat, including the microbiota as well as their genomes and the surrounding environment of the microbiota.

The phrase “topical composition” means a composition suitable for application directly to a surface, such as the surface of a human or animal body, including skin, and/or other surfaces, such as hair and nails.

The phrase “statistically significant” means p<0.05 for a test composition vs. a control that does not contain the active ingredient. The analysis is completed using 1) a T-test (a statistical examination of two population means) when only comparing one test article vs. one control); or 2) an analysis of variance (ANOVA) test when comparing two or more test articles vs. controls.

The general inventive concepts relate to a topical composition that contains an active ingredient that includes one or more of a probiotic, a probiotic-derived ingredient, and a prebiotic and/or prebiotic-derived ingredient. Generally, the active ingredient helps to restore skin's natural balance of bacteria and increase the production and/or activity of antimicrobial peptides. In some exemplary embodiments, the topical composition disclosed herein prevents pathogens from adhering to a surface, such as human skin or any inanimate surface. Such adherence prevention includes not only impeding the binding of a pathogen, but also promoting detachment of any already bound pathogen, and otherwise limiting the presence of such pathogens on a surface.

In some exemplary embodiments, the topical composition comprises one or more probiotics and/or probiotic-derived ingredients (probiotic derivatives). In general, the probiotic can be any living or dead microorganism that provides a health benefit to the host. The probiotic derivative can be any derivative of any type of probiotic. In some exemplary embodiments, the derivative is one or more of an excretion from a probiotic and a fragment of a probiotic. The fragment can be any portion of the probiotic microorganism including any portion of its DNA thereof.

Some non-limiting examples of probiotic and probiotic-derived ingredients include strains and derivates of the following families: Actinomycetaceae, Corynebacteriaceae, Nocardiaceae, Intrasporangiaceae, Micrococcaceae, Propionibacteriacea, Bacteroidaceae, Porphyromonadaceae, Flavobacteriaceae, Sphingobacteriaceae, Bacillaceae, Exiguobacteraceae, Gemellaceae, Planococcaceae, Staphlococcaceae, Carnobacteriaceae, Aeorcoccaceae, Lactobacillaceae, Acidaminacoccaceae, Clostridiaceae, Lachnospiraceae, Peptostreptococcaceae, Veillonellaceae, Caulobactereaceae, Acetobacteraceae, Rhodobacteriaceae, Bradyrhizobiaceae, Brucellaceae, Sphingomonadaceae, Comamonadaceae, Neisseriaceae, Enterobaceriaceae, Pseudomonodaceae, Moraxellaceae, Pasteurellaceae, Xanthomonadaceae, Fusobacteriaceae, Chloroflexi, Chloroplasts, Cyanobacteria, and Streptophyta, for example. In some exemplary embodiments, the active ingredient is a probiotic or probiotic derived ingredient, which can be selected from a strain of one or more the following: Lactobacillus, strains and derivatives of Clostridia, strains and derivatives of Bifidobacterium, strains and derivatives of Saccharomyces, strains and derivatives of Lactococcus, strains and derivatives of Pedicoccus, strains and derivatives of Enterococcus, strains and derivatives of Escherichia, strains and derivatives of Alcaligenes, strains and derivatives of Corynebacterium, strains and derivatives of Bacillus, and strains and derivatives of Propionibacterium.

In some exemplary embodiments, the probiotic or probiotic derived ingredient is a ferment of Bacillus coagulans. Bacillus is a genus of Gram-positive, rod-shaped bacteria of the phylum Fimicutes. Bacillus can be either aerobic or, under certain conditions, anaerobic and produces endospores. Bacillus exhibits a wide range of physiologic properties that allows it to thrive in a number of different habitats—most Bacillus strains are resistant to heat, cold, radiation, and disinfectants. A Bacillus ferment is sold under the trade name Bonicel™ by Ganeden Biotech, Inc. in Cleveland, Ohio and is the supernatant produced by Bacillus coagulans GBI-30, 6086 (collectively referred to herein as “Bonicel™”). Bonicel™ is produced though a fermentation process which ensures the formulation includes the maximum amounts of enzymes, bateriocins, and L+ Lactic acid. Additional probiotic or probiotic derived ingredients may include Repair Complex CLR™, EcoSkin® from Solabia Group, Leucidal® Liquid SF from Active Micro Technologies, ProSynergen™ from Lonza Group, ProBioBalance CLR™ from CLR, Yogurtene® Balance from Lonza Group, Biodynes™ from Lonza Group, and Bifidobacterium Longum Lysate.

In some exemplary embodiments, the active ingredient is one or more prebiotics and/or prebiotic derived ingredients (prebiotic derivatives). Generally, the prebiotic can be any compound that affects the ecology and/or environment of the microbiome by increasing good bacteria and/or decreasing bad bacteria. The prebiotic can affect the ecology and/or environment of the microbiome by, for example, feeding particular organisms, by altering oxygen levels, by changing temperature, by altering water content, by changing salinity, or by altering nutrient levels/types. Some non-limiting examples of prebiotic ingredients include alpha and beta-glucan oligosaccharides, trans-galactooligosaccharides, xylooligosaccharide, frutooligosaccharides, lactulose, ginseng, black current extract, sugar-beet extract, garlic extract, bark extract, chicory extract, corn extract, nerolidol extract, xylitol, and pectin. Additional prebiotic ingredients may include EmulGold™ Fibre by Kerry Ingredients, Genu® Explorer Pectin by CP Kelco, Orafti® from Beneo, VitaFiber™ from BioNeutra, Konjac Glucomannan Hydrolysates, and Oat Beta Glucan from VegeTech.

In some exemplary embodiments, the topical composition comprises a mixture of probiotics/probiotic derivatives and prebiotics/prebiotic derivatives as the active ingredient.

In some embodiments, the active ingredient functions to simulate the production and/or activity of antimicrobial peptides and thereby increase the overall concentration of AMPS on the surface of the skin. In some exemplary embodiments, the topical composition disclosed herein includes an effective amount of active ingredient to increase the production and/or activity of at least one antimicrobial peptide on, for example, the skin. The topical composition can increase the production and/or activity of a wide variety of antimicrobial peptides, such as, for example defensins and cathelicidin-related AMPs and decrease pro-inflammatory factors. Such increased production and/or activity helps the skin's ability to defend against germs and helps improve the skin's innate immunity. While topical compositions that can increase the skin's innate immunity or the production and/or activity on the skin are often discussed herein, it is to be appreciated that the topical compositions can provide the same benefits to nails, epithelial cells, as well as other parts of mammalian bodies.

The skin naturally produces AMPs, but the levels produced are not sufficient to produce the desired effect of long lasting germ defense and innate immunity on the skin. The active ingredient of the exemplary embodiments described herein has been found to help increase the production and/or activity of AMPs at levels significantly higher than the skin alone.

In one exemplary embodiment, the topical composition increases the production and/or activity of defensins. Defensins are cationic proteins that function as host defense peptides and have been found in vertebrates, invertebrates, and some plants. Defenins include at least α-defensins, β-defensins, and θ-defensins. In some exemplary embodiments, the topical composition increases the production and/or activity of β-defensins, such as HBD-2.

In some exemplary embodiments, the topical composition increases the production and/or activity of cathelicidin-related antimicrobial peptides. Cathelicidins play a vital role in mammalian innate immunity against invasive bacterial infections. In some exemplary embodiments, the topical composition increases the production and/or activity of the cathelcidin-related AMP, LL-37.

In other exemplary embodiments, the topical composition decreases the production and/or activity of pro-inflammatory factors. One such pro-inflammatory factor is cytokines, which are a group of small proteins that are involved in cell signaling. There are numerous groups of cytokines including chemokiens, interferons, interleukins, lymphokines, and tumor necrosis factors. Interleukins are one group of cytokines and include 17 different families, interleukins 1-17. In some exemplary embodiments, the topical composition increases the production and/or activity of the pro-inflammatory factor, cytokines. In some exemplary embodiments, the topical composition increases the production and/or activity of the cytokine, interleukins, such as interleukin-8 (IL-8).

In some exemplary embodiments, the topical composition increases the production and/or activity of cadherins. In some exemplary embodiments, the cadherins can be within the desmosomal class and within the desmocollin subclass of cadherins. Cadherins are type-1 trasmembrane proteins that are involved in cell adhesion, specifically adhesions junctions in binding cells one another. In this way, they are referred to herein as skin junction biomarkers. In some exemplary embodiments, the topical composition increases the production and/or activity of the skin junction biomarker, desmosomals, such as desmocollin-3 (DCS3).

Traditionally, it has been found that compositions used to stimulate the production and/or activity of AMPs also cause skin inflammation and/or skin irritation. However, it has been discovered that a topical composition comprising the subject active ingredient is capable of increasing the production and/or activity of at least one AMP on the skin without causing irritation/inflammation of the skin.

In some embodiments, the active ingredient helps to restore the microbial balance of bacteria on the skin. A human's skin microbiota includes resident skin microorganisms that are continuously present on the skin. The resident skin microorganisms are usually non-pathogenic and either commensals (not harmful to their host) or mutualistic (offer a benefit). Resident skin microorganisms are adapted to survive on skin and they eat, reproduce, and excrete, which has an effect on the skin. However, certain transient skin microorganisms may attempt to colonize the skin, which could upset a healthy microbiome. Such transient skin microorganisms may include pathogens, such as pathogenic bacteria, yeasts, viruses, and molds. The particular make-up of a human's microbiome may be different than the make-up of another human's. A resident skin microorganism on one person may be a transient on another.

While the skin naturally works to regulate the microbiota on the surface, the active ingredients disclosed herein have been found to help in regulating and restoring the skin's natural balance.

The topical composition may comprise up to about 15.0 weight percent (wt. %) of the active ingredient, or up to about 8.0 wt. %, or up to about 5.0 wt. %, or up to about 3.0 wt. %, or up to about 2.0 wt. % of the active ingredient, based on the total weight of topical composition. The topical composition may comprise at least about 0.001 wt. % of the active ingredient, or at least about 0.005 wt. %, or at least about 0.01 wt. %, or at least about 0.05 wt. %, or at least about 0.1 wt. %, or at least about 0.5 wt. %, or at least about 1.0 wt. % of the active ingredient, based on the total weight of the topical composition.

In some exemplary embodiments, the effective amount of active ingredient comprises from about 0.005 to about 15.0 wt. %, or from about 0.02 to about 5.0 wt. %, or from about 0.5 to about 2.0 wt. %, based on the total weight of the topical composition. In other exemplary embodiments, the effective amount of active ingredient comprises about 0.1 to about 1.0 wt. %, based on the total weight of topical composition. In one exemplary embodiment, the topical composition comprises about 0.08 to about 0.2 wt. % of the active ingredient, based on the total weight of topical composition.

In some exemplary embodiments, the topical composition is in the form of a cleanser, such as a soap or a lotion-based cleanser and is used for application to the skin. The topical composition may be in the form of a skin cleanser, skin moisturizer, skin protectant, shampoo, a wipe, a lotion, a salve, foam, soap, gel, a cream, etc. A wide variety of vehicles may be used to deliver the topical composition, such as, for example pads, bandages, patches, sticks, aerosol dispersers, pump sprays, trigger sprays, canisters, foam pumps, wipes, and the like. The topical composition may be applied to the skin before, during, or after skin cleaning.

In some exemplary embodiments, the topical composition comprises a carrier. The carrier can be any suitable compound able to effectively deliver and/or transport the topical composition. In some exemplary embodiments, the carrier is water or a base cleaner. In other exemplary embodiments, the topical composition does not include any carrier and is delivered as a concentrate.

In some exemplary embodiments, the topical composition includes water in an amount quantum sufficit (q.s.). In some exemplary embodiments, the topical composition comprises at least about 1.0 wt. % water, in another embodiment the topical composition comprises at least about 10.0 wt. % water, in another embodiment, the topical composition comprises at least about 20.0 wt. % water, in another embodiment, the topical composition comprises at least about 30.0 wt. % water, in another embodiment, the topical composition comprises at least about 40.0 wt. % water, in another embodiment, the topical composition comprises at least about 50.0 wt. % water, and in yet another embodiment, the topical composition comprises at least about 60.0 wt. % water, and in still yet another embodiment, the topical composition comprises at least about 70.0 wt. % water, based on the total weight of topical composition. In other embodiments, the topical composition comprises from about 20.0 wt. % to about 30.0 wt. % water, based on the total weight of topical composition. In yet other embodiments, the topical composition comprises from about 20.0 to about 24.0 wt. % water, based on the total weight of topical composition. More or less water may be required in certain instances, depending particularly on other ingredients and/or the amounts thereof employed in the topical composition.

In one or more embodiments, the topical composition includes one or more skin-conditioners. Various classes or types of skin-conditioners can be used such as humectants, emollients, and other miscellaneous compounds which exhibit occlusive properties upon application to the skin. Non-limiting examples of suitable skin conditioners and emollients include aloe, vitamin E, vitamin E acetate (tocopheryl acetate), Vitamin B3 (niacinamide), C₆₋₁₀ alkane diols, sodium salt of pyroglutamic acid (sodium PCA), PEG-7 glyceryl cocoate, coco-glucoside and/or glyceryl oleate (Lamisoft® PO), and polyquaternium, such as polyquaternium 10 and 39.

If an emollient or one of the miscellaneous skin-conditioners, such compound can be included in the topical composition in an amount from about 0.0001 to about 10.0 wt. %, in other embodiments, from about 0.0005 to about 5.0 wt. %, based on the total weight of the composition. In one exemplary embodiment, the miscellaneous skin conditioner is present in an amount from about 0.1 to about 2.0 wt. %, based on the total weight of topical composition and in yet another exemplary embodiment, from about 0.5 to about 1.0 wt. %, based on the total weight of topical composition.

In some exemplary embodiments, the topical composition includes one or more humectants as the skin conditioner. Non-limiting examples of humectants include propylene glycol, hexylene glycol, 1,4-dihydroxyhexane, 1,2,6-hexanetriol, sorbitol, butylene glycol, caprylyl glycol, propanediols, such as methyl propane diol, dipropylene glycol, triethylene glycol, glycerin (glycerol), polyethylene glycols, ethoxydiglycol, polyethylene sorbitol, glyceryl caprylate/caprate (GCC), and combinations thereof. Other humectants include glycolic acid, glycolate salts, lactate salts, urea, Jojoba wax PEG-120 esters (commercially available from FloraTech), hydroxyethyl urea, alpha-hydroxy acids, such as lactic acid, sodium pyrrolidone carboxylic acid, hyaluronic acid, chitin, and the like. In one exemplary embodiment, the humecant is a mixture of caprylyl glycol, sodium L-pyroglutamate (Sodium PCA), and glycerin.

Examples of polyethylene glycol humectants include PEG-4, PEG-6, PEG-7, PEG-8, PEG-9, PEG-10, PEG-12, PEG-14, PEG-16, PEG-18, PEG-20, PEG-32, PEG-33, PEG-40, PEG-45, PEG-55, PEG-60, PEG-75, PEG-80, PEG-90, PEG-100, PEG-135, PEG-150, PEG-180, PEG-200, PEG-220, PEG-240, and PEG-800.

The humectant may be included in the topical composition in an amount up to about 20.0 wt. %, or up to about 15.0 wt. %, or up to about 12.0 wt. %, or up to about 10.0 wt. %, or up to about 8.0 wt. %, or up to about 3.0 wt. %, based on the total weight of topical composition. In some exemplary embodiments, the humectant is included in an amount from about 0.001 wt. %, or from about 0.01 wt. %, or from about 0.05 wt. %, or from about 0.1 wt. %, or from about 0.5 wt. %, or from about 0.7 wt. %, or from about 1.0 wt. %, or from about 1.5 wt. %, or from about 2.0 wt. %, based on the total weight of topical composition. In one exemplary embodiment, the humectant is included in an amount from about 0.4 to about 3.0 wt. %, or from about 1.5 to about 2.0 wt. %, based on the total weight of topical composition.

In some exemplary embodiments, the topical composition further comprises a plug-preventing additive. In general, the additive prevents the hydroalcoholic gel from coagulating into solid or semi-solid material that may deposit onto a surface or plug a dispenser nozzle. In some exemplary embodiments, the plug-preventing additive can also, as discussed above, act as the humectant.

In one exemplary embodiments, the plug-preventing additive comprises a hydrocarbon chain with two or more carbon atoms. The hydrocarbon can be branched or straight and can also be cyclic or linear. The hydrocarbon can have any number of various functional groups including, but not limited to, amines, esters, carboxylic acids, ethers, amides, alkyl halides, alcohols, phenyls, as well as other carbonyl-containing functional groups. The hydrocarbon molecule can be anionic, cationic, or non-ionic.

In one exemplary embodiment, the hydrocarbon contains one or more esters. In some exemplary embodiments, the plug-preventing additive comprises a monomeric or polymeric di-ester, tri-ester, tetra-ester, penta-ester, or hexa-ester, or a polymeric monoester. In one or more embodiments, the plug-preventing additive includes one or more of C₁-C₃₀ alcohol esters of C₁-C₃₀ carboxylic acids, ethylene glycol monoesters of C₁-C₃₀ carboxylic acids, ethylene glycol diesters of C₁-C₃₀ carboxylic acids, propylene glycol monoesters of C₁-C₃₀ carboxylic acids, propylene glycol diesters of C₁-C₃₀ carboxylic acids, C₁-C₃₀ carboxylic acid monoesters and polyesters of polypropylene glycols, C₁-C₃₀ carboxylic acid monoesters and polyesters of polypropylene glycols, C₁-C₃₀ carboxylic acid monoesters and polyesters of C₄-C₂₀ alkyl ethers, C₁-C₃₀ carboxylic acid monoesters and polyesters of di-C₈-C₃₀ alkyl ethers, and mixtures thereof.

Non-limiting examples of plug-preventing additives with esters include acetyl tributyl citrate, acetyl triethyl citrate, acetyl triethylhexyl citrate, acetyl trihexyl citrate, butyl benzyl phthalate, butyl phthalyl butyl glycolate, butyroyl trihexyl citrate, dibutyl adipate, dibutyloctyl malate, dibutyl oxalate, dibutyl phthalate, dibutyl sebacate, dicapryl adipate, dicaprylyl/capryl sebacate, diethylene glycol dibenzoate, diethylene glycol di ethyl hexanoate/diisononanoate, diethylene glycol diisononanoate, diethylene glycol rosinate, diethylhexyl adipate, diethylhexyl phthalate, diethylhexyl sebacate, diethylhexyl succinate, di ethylhexyl terephthalate, diethyl oxalate, diethyl phthalate, diethyl sebacate, diethyl succinate, diisoamyl malate, diisobutyl adipate, diisobutyl maleate, diisobutyl oxalate, diisocetyl adipate, diisocetyl dodecanedioate, diisodecyl adipate, diisononyl adipate, diisocetyl adipate, diisooctyl maleate, diisooctyl sebacate, diisopropyl adipate, diisopropyl oxalate, diisopropyl sebacate, diisopropyl dimer dilinoleate, diisostearyl adipate, diisostearyl fumarate, diisostearyl glutarate, diisostearyl malate, diisostearyl sebacate, dimethyl adipate, dimethyl oxalate, dimethyl phthalate, dioctyldodecyl adipate, Dioctyldodecyl Dimer Dilinoleate, Dioctyldodecyl Dodecanedioate, Dioctyldodecyl Fluoroheptyl Citrate, Dioctyldodecyl IPDI, Dioctyldodecyl Lauroyl Glutamate, Dioctyldodecyl Malate, Dioctyldodecyl Sebacate, Dioctyldodecyl Stearoyl Glutamate, dipentaerythrityl hexa C₅₋₉ acid esters, dipentaerythrityl hexa C₅₋₁₀ acid esters, dipropyl oxalate, pentaerythrityl tetra C₅₋₉ acid esters, pentaerythrityl tetra C₅₋₁₀ acid esters, tributyl citrate, tricaprylyl/capryl trimellitate, triethyl citrate, triethylene glycol dibenzoate, triethylene glycol rosinate, triethylhexyl citrate, triethylhexyl trimellitate, trimethylpentanediyl dibenzoate, trimethyl pentanyl diisobutyrate, polyglyceryl-6 pentacaprylate, polyglyceryl-10 pentahydroxystearate, polyglyceryl-10 pentaisostearate, polyglyceryl-10 pentalaurate, polyglyceryl-10 pentalinoleate, polyglyceryl-5 pentamyristate, polyglyceryl-4 pentaoleate, polyglyceryl-6 pentaoleate, polyglyceryl-10 pentaoleate, polyglyceryl-3 pentaricinoleate, polyglyceryl-6 pentaricinoleate, polyglyceryl-10 pentaricinoleate, polyglyceryl-4 pentastearate, polyglyceryl-6 pentastearate, polyglyceryl-10 pentastearate, sorbeth-20 pentaisostearate, sorbeth-30 pentaisostearate, sorbeth-40 pentaisostearate, sorbeth-50 pentaisostearate, sorbeth-40 pentaoleate, sucrose pentaerucate, and triacetin, combinations thereof. In some exemplary embodiments the hydrocarbon plug-preventing additive is selected from one or more of isopropyl myristate and diisopropyl sebacate.

In one or more embodiments, the plug-preventing additive comprises a polymeric ester. The polymeric ester can include one or more ester groups.

In some exemplary embodiments, the polymer chain includes a polyethylene glycol (PEG) chain, a polypropylene glycol (PPG), or a combination thereof. In one or more embodiments, the polymer chain includes up to about 12 PEG units, PPG units, or a combination thereof. In some exemplary embodiments, the polymer chain includes up to about 10 PEG units, PPG units, or a combination thereof. In some exemplary embodiments, the polymer chain includes up to about 8 PEG units, PPG units, or a combination thereof. In some exemplary embodiments, the polyether polymer chain includes from about 1 to about 12 PPG or PEG units, or from about 2 to about 8 PPG or PEG units, or a combination thereof.

Examples of polymeric esters include those that may be represented by the following formula

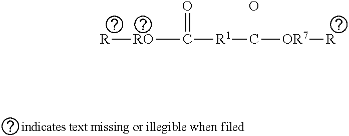

wherein R¹ is a linear or branched alkyl group having from 1 to 28 carbon atoms, each R², which may be the same or different, includes a polyether chain having up to about 12 PEG or PPG groups, or a combination thereof, and each R³, which may be the same or different, includes an alkyl or alkylene group having from 1 to about 30 carbon atoms, and wherein each R³ group is attached to R² via an ether linkage.

In some exemplary embodiments, R¹ includes up to about 20 carbon atoms, or up to about 10 carbon atoms, or up to about 8 carbon atoms. In some exemplary embodiments, R³ may be represented by the formula CH₃(CH₂)_{z}O—, wherein z is an integer from 1 to about 21, or from 2 to about 17, or from 3 to about 15.

In one or more embodiments, the polymeric ester may be represented by the following formula

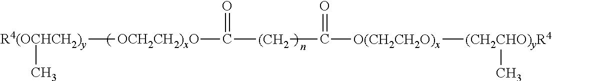

wherein R⁴ includes a linear or branched, alkyl or alkylene group having from 1 to about 22 carbon atoms. In some exemplary embodiments, R⁴ may be represented by the formula CH₃(CH₂)_{z}—, wherein some exemplary embodiments z is an integer from 1 to about 21, or from 2 to about 17, or from 3 to about 15. In some exemplary embodiments, n is an integer from 1 to about 20, or from 2 to about 10. In some exemplary embodiments x is an integer up to about 12, or up to about 10, or up to about 8, or is zero. In some exemplary embodiments, y is an integer up to about 12, or up to about 10, or up to about 8, or is zero. Examples of polymeric esters further include those that may be represented by the following formula

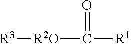

wherein R¹, R², and R³ are as described hereinabove.

Examples of polymeric esters include any of the above di-, tri, tetra-, penta-, or hexa-esters modified to include a PPG, PEG, or PPG/PEG polymer chain of the appropriate length. Specific examples include Di-PPG-3-ceteth-4 adipate, Di-PPG-2-myreth-10 adipate, Di-PPG-3-myristyl ether adipate, and PPG-2 myristyl ether propionate. In some exemplary embodiments, a mixture of one or more polymeric esters and one or more monomeric di-, tri-, tetra-, penta-, or hexa-esters may be employed as plug-preventing additives.

In other exemplary embodiments the plug-preventing comprises one or more diols, that is compounds with two hydroxyl groups. Plug-preventing additives that contain more or less hydroxyl groups (i.e., one hydroxyl group and three or more hydroxyl groups) are also within the purview of the exemplary embodiments disclosed herein. In one or more exemplary embodiments the diol is a C₆₋₁₀ alkane diol and/or a straight chain C₆₋₁₀ alkane diol. Non-limiting examples of suitable diols include 1,2-hexanediol, 1,2-octanediol (often referred to as caprylyl glycol), 1,9-nonanediol, 1,2-decanediol, 1,10-decanediol, or mixtures and blends thereof. It is envisioned that the diol can contain any other functional groups including, for example, esters, carboxylic acids, ethers, amides, amines, alkyl halides, phenyls, as well as other carbonyl-containing functional groups. In some exemplary embodiments, the plug-preventing agent contains at least one ester and/or at least one amide group. In some exemplary embodiments, the plug-preventing agent is selected from glyceryl caprylate/caprate (GCC) and cocoamide diethanolamine.

If separate from the humectant, the plug-preventing additive may be included in the topical composition in an amount up to about 20.0 wt. %, or up to about 15.0 wt. %, or up to about 12.0 wt. %, or up to about 10.0 wt. %, or up to about 8.0 wt. % or up to about 5.0 wt. %, or up to about 3.0 wt. %, based on the total weight of the topical composition. In some exemplary embodiments, the plug-preventing agent is included in an amount from about 0.001 wt. %, or from about 0.01 wt. %, or from about 0.05 wt. %, or from about 0.1 wt. %, or from about 0.5 wt. %, or from about 0.7 wt. %, or from about 1.0 wt. %, or from about 1.5 wt. %, or from about 2.0 wt. %, based on the total weight of the topical composition. In one exemplary embodiment, the plug-preventing additive is included in an amount from about 0.05 to about 4.0 wt. %, or from about 0.1 to about 1.0 wt. %, or from about 0.15 to about 0.7 wt. %, or from about 0.2 to about 0.7 wt. %, based on the total weight of the topical composition.

In certain embodiments, the plug-preventing additive is added to the composition as a solution or emulsion. That is, the plug-preventing additive can be premixed with a carrier to form a solution or emulsion, with the proviso that the carrier does not deliriously effect the ability of the sanitizing composition to sanitize and kill non-enveloped viruses. Examples of carriers include water, alcohol, glycols such as propylene or ethylene glycol, ketones, linear and/or cyclic hydrocarbons, triglycerides, carbonates, silicones, alkenes, esters such as acetates, benzoates, fatty esters, glyceryl esters, ethers, amides, polyethylene glycols and PEG/PPG copolymers, inorganic salt solutions such as saline, and mixtures thereof. It will be understood that, when the plug-preventing additive is premixed to form a plug-preventing additive solution or emulsion, the amount of solution or emulsion that is added to the topical composition is selected so that the amount of plug-preventing additive falls within the ranges set forth hereinabove.

The topical composition may further comprise one or more conditioning or moisturizing esters. Examples of such conditioning or moisturizing esters include cetyl myristate, cetyl myristoleate, and other cetyl esters, diisopropyl sebacate, and isopropyl myristate. The ester may be present in an amount of up to about 10.0 wt. %, or up to about 8.0 wt. %, or up to about 5.0 wt. %, or up to about 3.0 wt. %, or up to about 2.0 wt. %, or up to about 1.0 wt. %, based on the total weight of topical composition. In some exemplary embodiments, the moisturizing ester is present in an amount from about 0.001 wt. %, or from about 0.005 wt. %, or from about 0.01 wt. %, or from about 0.05 wt. %, or from about 0.1 wt. %, or from about 0.5 wt. %, or from about 1.0 wt. %, based on the total weight of the topical composition. In one exemplary embodiment, the moisturizing ester is present in an amount between 0.01 to 0.3 wt. %, based on the total weight of the composition. In another exemplary embodiment, the moisturizing ester is present in an amount between 0.05 wt. % and 0.25 wt. %, based on the total weight of topical composition.

The topical composition may further comprise one or more deposition enhancers. A suitable deposition enhancer works unidirectionally and will allow ingredients within the composition to penetrate deeper into the stratum corneum whilst preventing the loss of materials from the skin. Advantageously, the deposition enhancer provides a cosmetically acceptable skin feel to the formulation.

In one or more embodiments, the deposition enhancers include one or more of surfactants, bile salts and derivatives thereof, chelating agents, and sulphoxides.

Some examples of acceptable deposition enhancers include hydroxypropyl methylcellulose, dimethyl sulphoxides (DMSO), DMA, DMF, 1-dodecylazacycloheptan-2-one (azone), pyrrolidones such as 2-Pyrrolidone (2P) and N-Methyl-2-Pyrrolidone (NMP), long-chain fatty acids such as oleic acid and fatty acids with a saturated alkyl chain length of about C₁₀-C₁₂, essential oils, terpenes, terpenoids, oxazolidinones such as 4-decyloxazolidin-2-one, sodium lauryl sulfate (SLS), sodium laureate, polysorbates, sodium glyacolate, sodium deoxycholate, caprylic acid, EDTA, phospholipids, C₁₂₋₁₅ Alkyl Benzoate, pentylene glycol, ethoxydiglycol, polysorbate-polyethylenesorbitan-monolaurate, and lecithin.

In one or more exemplary embodiments, the deposition enhancer is a quaternary ammonium compound such as polyquaternium-6, -7, -10, -22, -37, -39, -74 or -101.

The deposition enhancer may be included in the topical composition in an amount from about 0.005 wt. % to about 10.0 wt. %, in other embodiments, from about 0.01 wt. % to about 5.0 wt. %, and in other embodiments, from about 0.05 wt. % to about 3.0 wt. %, based on the total weight of the composition.

In one or more exemplary embodiments, the deposition enhancer comprises a hydroxy-terminated polyurethane compound chosen from polyolprepolymer-2, polyolprepolymer-14, and polyolprepolymer-15. Polyolprepolymer-2 is sometimes referred to as PPG-12/SMDI copolymer. The polyurethane compound may be present in the topical composition in an amount from about 0.005 wt. % to about 5.0 wt. %, in other embodiments, from about 0.01 wt. % to about 3.0 wt. %, and in other embodiments, from about 0.05 wt. % to about 1.0 wt. %, based on the total weight of topical composition.

The topical composition may further comprise one or more preservatives. A preservative is a natural or synthetic ingredient that can be added to personal care products to prevent spoilage, such as from microbial growth or undesirable chemical changes. Typical cosmetic preservatives are classified as natural antimicrobials, broad-spectrum preservatives, or stabilizers.

Many different types of preservatives are envisioned as being applicable in the current topical composition. Non-limiting examples of preservatives include one or more of isothiazolinones, such as methylchloroisothiazolinone and methylisothiazolinone; parabens including butylparaben, propylparaben, methylparaben and germaben II; phenoxyetyhanol and ethylhexylglycerin, organic acids such as potassium sorbate, sodium benzoate and levulinic acid; and phenoxyethanols.

The preservative can be added in the topical composition in an amount up to about 10.0 wt. %, preferably from about 0.05 wt. % to about 5.0 wt. %, more preferably from about 0.1 wt. % to about 2.0 wt. %, based on the weight of the total composition. In one exemplary embodiment, the preservative is present in an amount from about 1.0 to about 1.5 wt. %, based on the total weight of topical composition.

The topical composition may further comprise one or more anti-irritants. Anti-irritants reduce signs of inflammation on the skin such as swelling, tenderness, pain, itching, or redness. There are three main types of anti-irritants, all of which are envisioned as being applicable in the exemplary embodiments described herein: (1) compounds that operate by complexing the irritant itself, (2) compounds that react with the skin to block reactive sites preventing the irritant from reacting directly with the skin, and (3) compounds that prevent physical contact between the skin and irritant.

Some exemplary examples of suitable anti-irritants include Aloe Vera, allantoin, anion-cation complexes, aryloxypropionates, azulene, carboxymethyl cellulose, cetyl alcohol, diethyl phthalate, Emcol E607, ethanolamine, glycogen, lanolin, N-(2-Hydroxylthyl) Palmitamide, N-Lauroyl Sarcosinates, Maypon 4C, mineral oils, miranols, Myristyl lactate, polypropylene glycol, polyvinyl pyrrolidone (PVP), tertiary amine oxides, thiodioglycolic acid, and zirconia. In one exemplary embodiment, the anti-irritant is avenanthrmides (avena sativa (oat), kernel oil, and glycerin) and niacinamide.

The anti-irritant may be included in the topical composition in an amount up to about 10.0 wt. %, in other embodiments, from about 0.005 wt. % to about 3.0 wt. %, and in other embodiments, from about 0.01 wt. % to about 1.0 wt. %, based on the total weight of topical composition.

The topical composition may further comprise a fragrance. Any scent may be used in the topical composition including, but not limited to, any scent classification on a standard fragrance chart, such as floral, oriental, woody, and fresh. Exemplary scents include cinnamon, clove, lavender, peppermint, rosemary, thyme, thieves, lemon, citrus, coconut, apricot, plum, watermelon, ginger and combinations thereof.

The fragrance can be included in the topical composition in an amount from about 0.005 wt. % to about 5.0 wt. %, in other embodiments, from about 0.01 wt. % to about 3.0 wt. %, and in other embodiments, from about 0.05 wt. % to about 1.0 wt. %, based on the total weight of topical composition. The fragrance can be any made of any perfume, essential oil, aroma compounds, fixatives, terpenes, solvents, and the like. In some exemplary embodiments, the essential oils may include, for example, one or more of Limonene, Citrus Aurantium Dulcis (Orange) Peel Oil, Eucalyptus Globulus Leaf Oil, Citrus Grandis (Grapefruit) Peel Oil, Linalool, Litsea Cubeba Fruit Oil, Lavandula Hybrida Oil, Abies Sibirica Oil, Mentha Citrata Leaf Extract, Coriandrum Sativum (Coriander) Fruit Oil, Piper Nigrum (Pepper) Fruit Oil, and Canarium Luzonicum Gum Nonvolatiles.

The topical composition may further comprise a wide range of optional ingredients that do not deleteriously affect the composition's ability to stimulate AMP production and/or activity and that do not deleteriously affect the composition's ability to restore the microbial balance on the surface of the skin. The CTFA International Cosmetic Ingredient Dictionary and Handbook, Eleventh Edition 2005, and the 2004 CTFA International Buyer's Guide, both of which are incorporated by reference herein in their entirety, describe a wide variety of non-limiting cosmetic and pharmaceutical ingredients commonly used in the skin care industry, that are suitable for use in the compositions of the exemplary embodiments described herein. Examples of these functional classes include: abrasives, anti-acne agents, anticaking agents, antioxidants, binders, biological additives, bulking agents, chelating agents, chemical additives; colorants, cosmetic astringents, cosmetic biocides, denaturants, drug astringents, emulsifiers, external analgesics, film formers, fragrance components, opacifying agents, plasticizers, preservatives (sometimes referred to as antimicrobials), propellants, reducing agents, skin bleaching agents, skin-conditioning agents (emollient, miscellaneous, and occlusive), skin protectants, solvents, surfactants, foam boosters, hydrotropes, solubilizing agents, suspending agents (nonsurfactant), sunscreen agents, ultraviolet light absorbers, detackifiers, and viscosity increasing agents (aqueous and nonaqueous). Examples of other functional classes of materials useful herein that are well known to one of ordinary skill in the art include solubilizing agents, sequestrants, keratolytics, topical active ingredients, and the like.

The inventive coating compositions exhibit a pH in the range of from about 2.5 to about 12.0, or a pH in the range of from about 3.5 to about 8.5, or in the range of from about 4.0 and about 8.0. When necessary, a pH adjusting agent or constituent may be used to provide and/or maintain the pH of a composition. Exemplary pH adjusting agents include, but are not limited to, organic acids, such as citric acid, lactic acid, formic acid, acetic acid, propionic acid, butyric acid, caproic acid, oxalic acid, maleic acid, benzoic acid, carbonic acid, and the like.

The form of the composition of the exemplary embodiments described herein is not particularly limited. In one or more embodiments, topical compositions of the exemplary embodiments described herein may be formulated as a lotion, a foamable composition, a rinse-off soap composition, a thickened gel composition, or may be applied to a wipe.

In one or more embodiments, the topical composition is formulated as a foamable composition. One or more foam agents may optionally be included in the foamable composition.

Any foaming agent conventionally known and used may be employed in the topical composition. In one or more embodiments, the foam agent comprises a non-ionic foam agent such as decyl glucoside or an amphoteric foam agent such as cocamidopropylbetaine. In one or more embodiments, the amount of nonionic or amphoteric foam agent is from about 0.5 to about 3.5 wt. %, in other embodiments from about 1.0 to about 3.0 wt. %, based on the total weight of the topical composition. In one or more embodiments, the amount of decyl glucoside or cocamidopropylbetaine is from about 0.5 to about 3.5 wt. %, in other embodiments from about 1.0 to about 3.0 wt. %, based on the total weight of the topical composition.

In some exemplary embodiments, the foaming agents include one or more of silicone glycol and fluorosurfactants. Silicone glycols may be generally characterized by containing one or more Si—O—Si linkages in the polymer backbone. Silicone glycols include organopolysiloxane dimethicone polyols, silicone carbinol fluids, silicone polyethers, alkylmethyl siloxanes, amodimethicones, trisiloxane ethoxylates, dimethiconols, quaternized silicone glycols, polysilicones, silicone crosspolymers, and silicone waxes.

Examples of silicone glycols include dimethicone PEG-7 undecylenate, PEG-10 dimethicone, PEG-8 dimethicone, PEG-12 dimethicone, perfluorononylethyl carboxydecal PEG 10, PEG-20/PPG-23 dimethicone, PEG-11 methyl ether dimethicone, bis-PEG/PPG-20/20 dimethicone, silicone quats, PEG-9 dimethicone, PPG-12 dimethicone, fluoro PEG-8 dimethicone, PEG-23/PPG-6 dimethicone, PEG-20/PPG-23 dimethicone, PEG 17 dimethicone, PEG-5/PPG-3 methicone, bis-PEG-18 methyl ether dimethyl silane, bis-PEG-20 dimethicone, PEG/PPG-20/15 dimethicone copolyol and sulfosuccinate blends, PEG-8 dimethicone\dimmer acid blends, PEG-8 dimethicone\fatty acid blends, PEG-8 dimethicone\cold pressed vegetable oil\polyquaternium blends, random block polymers and mixtures thereof.

The amount of silicone glycol foam agent is not particularly limited, so long as an effective amount to produce foaming is present. In certain embodiments, the effective amount to produce foaming may vary, depending upon the amount of other ingredients that are present. In one or more embodiments, the composition includes at least about 0.002 wt. % of silicone glycol foam agent, based on the total weight of the composition. In another embodiment, the composition includes at least about 0.01 wt. % of silicone glycol foam agent, based on the total weight of topical composition. In yet another embodiment, the composition includes at least about 0.05 wt. % of silicone glycol foam agent, based on the total weight of topical composition.

In some exemplary embodiments, the foam agent is present in an amount of from about 0.002 to about 4.0 wt. %, or in an amount of from about 0.01 to about 2.0 wt. %, based on the total weight of topical composition. It is envisioned that higher amounts may also be effective to produce foam. All such weights as they pertain to listed ingredients are based on the active level, and therefore, do not include carriers or by-products that may be included in commercially available materials, unless otherwise specified.

In other embodiments, it may be desirable to use higher amounts of foam agent. For example, in certain embodiments where the foaming composition of the exemplary embodiments described herein includes a cleansing product that is applied to a surface and then rinsed off, higher amounts of foam agent may be employed. In these embodiments, the amount of foam agent is present in amounts up to about 35.0 wt. %, based on the total weight of topical composition.

The topical composition of the exemplary embodiments described herein may be formulated as an aerosol or non-aerosol foamable composition. In some exemplary embodiments the topical composition is dispensed from an unpressurized or low-pressure dispenser which mixes the composition with air.

In one or more embodiments, the viscosity of the non-aerosol foamable composition is less than about 100 mPas, in one embodiment less than about 50 mPas, and in another embodiment less than about 25 mPas.

In one or more embodiments, the compositions of the exemplary embodiments described herein may be formulated as a lotion. As is known in the art, lotions include oil-in-water emulsions as well as water-in-oil emulsions, oil-water-oil, and water-oil-water. A wide variety of ingredients may be present in either the oil or water phase of the emulsion. That is, the lotion formulation is not particularly limited.

Compositions of the exemplary embodiments described herein may be characterized by reference to viscosity and/or rheological properties. In one or more embodiments, the viscosity may be expressed as a standard, single-point type viscosity, as measured on a Brookfield Digital viscometer at a temperature of about 20° C., using spindle T-D, heliopath, at a speed of 10 rpm. In one or more embodiments, the compositions may have a viscosity of from about 2000 to about 120,000 centipoise (cP).

In one or more embodiments, compositions of the exemplary embodiments described herein may be characterized as lotions, having a viscosity of less than about 120,000 cP, in other embodiments, less than about 100,000, and in other embodiments, less than about 75,000 cP. In one or more embodiments, the lotion compositions may have a viscosity of from about 3000 to about 50,000 cP, in other embodiments, from about 4000 to about 30,000 cP.

Exemplary lotion formulations include those containing water and/or alcohols and emollients such as hydrocarbon oils and waxes, silicone oils, hyaluronic acid, vegetable, animal or marine fats or oils, glyceride derivatives, fatty acids or fatty acid esters or alcohols or alcohol ethers, lanolin and derivatives, polyhydric alcohols or esters, wax esters, sterols, phospholipids and the like, and generally also emulsifiers (nonionic, cationic or anionic), although some of the emollients inherently possess emulsifying properties.

In one or more embodiments, compositions of the exemplary embodiments described herein may be characterized as serum, having a viscosity of from about 2000 to about 3000 cP.

In one or more embodiments, compositions of the exemplary embodiments described herein may be characterized as creams, having a viscosity of from about 30,000 to about 100,000 cP, in other embodiments from about 50,000 to about 80,000 cP.

In one or more embodiments, compositions according to the exemplary embodiments described herein are pourable at room temperature, i.e. a temperature in the range of from about 20 to about 25° C. In one or more embodiments, the lotion formulations are viscous enough to hold a shape or not flow for a desired period of time. In other embodiments, compositions of the exemplary embodiments described herein are creams or ointments, and are not pourable and do not flow at room temperature and will not conform to a container when placed into the container at room temperature.

In one or more embodiments, the topical composition of the exemplary embodiments described herein may include thickeners and optionally one or more stabilizers. Examples of thickeners and stabilizers include polyurethane-based thickeners, such as steareth-100/PEG-136/HDI copolymer (Rheoluxe® 811); sodium chloride; propylene glycol; PEG-120 methyl glucose dioleate and methyl gluceth-10 (Ritathix DOE, available from Rita Corp.); hydroxyethyl cellulose; quaternized hydroxyethyl cellulose (Polyquaternium-10); hydroxypropyl cellulose; methyl cellulose; carboxymethyl cellulose; and ammonium acryloyldimethyltaurate/VP copolymer.

In one or more exemplary embodiments, the topical composition may be thickened with polyacrylate thickeners such as those conventionally available and/or known in the art. Examples of polyacrylate thickeners include carbomers, acrylates/C 10-30 alkyl acrylate crosspolymers, copolymers of acrylic acid and alkyl (C₅-C₁₀) acrylate, copolymers of acrylic acid and maleic anhydride, and mixtures thereof. In one or more embodiments, the gel composition includes an effective amount of a polymeric thickener to adjust the viscosity of the gel to a viscosity range of from about 1000 to about 65,000 cP. In one embodiment, the viscosity of the gel is from about 5000 to about 35,000 cP, and in another embodiment, the viscosity is from about 10,000 to about 25,000 cP. The viscosity is measured by a Brookfield RV Viscometer using RV and/or LV Spindles at 22° C.+/−3° C.

As will be appreciated by one of skill in the art, the effective amount of thickener will vary depending upon a number of factors, including the amount of other ingredients in the topical composition. In one or more embodiments, an effective amount of thickener is at least about 0.01 wt. %, based on the total weight of topical composition. In other embodiments, the effective amount is at least about 0.02 wt. %, or at least about 0.05 wt. %, or at least about 0.1 wt. %, based on the total weight of topical composition. In some exemplary embodiment, the effective amount of thickener is at least about 0.5 wt. %, or at least about 0.75 wt. %, based on the total weight of topical composition. In one or more embodiments, the compositions according to the exemplary embodiments described herein comprise up to about 10 wt. % of a polymeric thickener, based on the total weight of topical composition. In certain embodiments, the amount of thickener is from about 0.01 to about 1.0 wt. %, or from about 0.02 to about 0.4 wt. %, or from about 0.05 to about 0.3 wt. %, based on the total weight of topical composition. The amount of thickener may be from about 0.1 to about 10.0 wt. %, or from about 0.5 to about 5.0 wt. %, or from about 0.75 to about 2.0 wt. %, based on the total weight of topical composition.

In one or more embodiments, the topical composition may further comprise a neutralizing agent. Examples of neutralizing agents include amines, alkanolamines, alkanolamides, inorganic bases, amino acids, including salts, esters and acyl derivatives thereof. Exemplary neutralizing agents include triethanolamine, sodium hydroxide, monoethanolamine and dimethyl stearylamine. Other neutralizing agents are also known, such as HO(CₘH₂ₘ)₂NH, where m has the value of from 2 to 3, and aminomethyl propanol, aminomethyl propanediol, and ethoxylated amines, such as PEG-25 cocamine, polyoxyethylene (5) cocamine (PEG-5 cocamine), polyoxyethylene (25) cocamine (PEG-25 cocamine), polyoxyethylene (5) octadecylamine (PEG-5 stearamine), polyoxyethylene (25) octadecylamine (PEG-25 stearamine), polyoxyethylene (5) tallowamine (PEG-5 tallowamine), polyoxyethylene (15) oleylamine (PEG-15 oleylamine), polyethylene (5) soyamine (PEG-5 soyamine), and polyoxyethylene (25) soyamine (PEG-15 soyamine). A number of these are commercially available under the trade name of Ethomeen® from Akzo Chemie America, Armak Chemicals of Chicago, Ill.

In some exemplary embodiments the neutralizing agent includes at least one of sodium hydroxide or sodium hydroxide precursors. Solutions of sodium hydroxide in water are non-limiting examples of neutralizers containing sodium hydroxide.

The neutralizing agent is employed in an effective amount to neutralize a portion of the carboxyl groups of the thickening agent, and produce the desired pH range. The pH of un-neutralized thickening agent dispersed in water is generally acidic. For example, the pH of Carbopol® polymer dispersions is approximately in the range of 2.5 to 3.5, depending upon the polymer concentration. An effective amount of neutralizing agent, when added to the thickener dispersion, adjusts the pH to a desired range of about 4.1 to 4.8, or of about 4.2 to 4.6. The amount of neutralizing agent necessary to effect this pH range will vary depending upon factors such as the type of thickening agent, the amount of thickening agent, etc. However, in general, amounts less than 1.0 wt. % or ranging from about 0.001 to about 0.3 wt. % of the neutralizing agent, based on the total weight of topical composition are considered sufficient and effective.

In some exemplary embodiments the topical composition can also be formulated as a soap. A fatty acid or a fatty acid ester may be used in conjunction with an alkali or base from the water phase to form a soap which has good water solubility as well as oil solubility properties and hence, is an excellent emulsifier. The soap, as explained above, can be in the form of a lotion soap, a foam soap, or any other common form known to one of skill in the art. Typical commercial blends such as oleic fatty acid, coconut fatty acid, soya fatty acid and tall oil fatty acid can be used. Preferably, the fatty acid comprises from about 5.0 to about 10.0 wt. % of the total topical composition.

As explained above, a base may be utilized in conjunction with the fatty acid to produce a soap on an equivalent basis of from about 2.7 to 0.8 equivalents to 1 equivalent of base. Examples of suitable base include organic alkalis or amines such as monoethanolamine, triethanolamine, and mixed isopropanolamines such as diisopropanolamine. Examples of suitable base also include inorganic alkalis, such as potassium hydroxide, sodium hydroxide, ammonium hydroxide, soda ash, and ammonia.

In addition, one or more non-fatty acid soap surfactants can be included in the oil phase of the cleaning composition in amounts preferably ranging up to about 25.0 wt. %, based on the total weight of topical composition. A surfactant is generally any substance which reduces the surface tension of a liquid. They break down the interface between water and oils/dirt. By holding the oils/dirt in suspension, they can be easily removed from the surface (i.e. skin).

In some exemplary embodiments, the surfactant includes a mixture of primary and secondary surfactants. Nonionic surfactants, i.e., surfactants which are uncharged (neutral) and without cationic or anionic sites, are preferred since they tend to render the composition stable, i.e., impart two desirable properties thereto. The first property is that of a suitable long shelf life. In other words, the emulsion can be held together at room temperature for long periods of time. The second desirable property is that upon use of the cleaning composition, the surfactant permits breakage of the emulsion or opening up thereof such that the hydrocarbon oil is readily released. The surfactant can also be an anionic surfactant, which carry a negative charge and are ionized in solution. The surfactant can also be a cationic surfactant, which carry a positive charge and ionize in solution. The surfactant can also be an amphoteric surfactant, which have the ability to be anionic (negatively charged), cationic (positively charged), or nonionic (uncharged, neutral) in solution depending on the pH.

It will be appreciated by one skilled in the art that in one or more embodiments, surfactant and/or surfactant combinations may be chosen to limit irritation of the composition and/or to enhance the effect of the active ingredient. In yet another embodiment, surfactant and/or surfactant combinations may be chosen to allow maximum bioavailability of the active ingredient. Non-limiting exemplary examples of surfactant combinations, levels of which will be known to one skilled in the art, are sodium lauryl ether sulfate (SLES) and/or cocamidopropyl betaine and/or disodium cocoamphodiacetate and/or surfactants of similar structure.

Non-limiting exemplary examples of surfactants that are envisioned in the present composition include betaines such as cocamidoproyl betaine; sulfonates and sulfates such as sodium laureth sulfate, sodium cocosulfate, sodium trideceth sulfate, and alkylbenzene sulfonate; glucosides, such as lauryl gluocoside and decyl glucoside; sodium cocoyl isothionate, sodium cocoyl glycinate, cocamidopropyl hydroxysultaine, PEG-80 sorbitan laurate, di-alkyl sulfosuccinate, lignosulfonates, disodium cocoamphodiacetate, lauryl glucoside, and PEG-80 sodium laurate.

In some exemplary embodiments, the topical cleansing composition comprises at least one primary surfactant and at least one secondary surfactant. A primary surfactant may include, for example, sodium laureth sulfate. Exemplary secondary surfactants may include, for example, one or more of cocamidopropyl betaine, disodium cocoamphodiacetate, cocamidopropyl hydroxysultaine, and lauryl glucoside.

As will be appreciated by one of skill in the art, the amount of surfactant will vary depending upon a number of factors, including the amount of other ingredients in the topical composition. In some exemplary embodiments, the surfactant is included in at least about 0.5 wt. %, or at least about 0.75 wt. %, or at least about 1.0 wt. %, or at least about 2.0 wt. %, based on the total weight of topical composition. In one or more exemplary embodiments, the compositions according to the exemplary embodiments described herein comprise up to about 25.0 wt. %, or up to about 18.0 wt. %, or up to about 15.0 wt. %, or up to about 12.0 wt. %, or up to about 9.0 wt. %, based on the total weight of topical composition of one or more surfactants. In certain exemplary embodiments, the amount of surfactant is from about 2.0 wt. % to about 20.0 wt. %, or from about 2.5 wt. % to about 18.0 wt. %, or from about 3.0 wt. % to about 13.0 wt. %, based on the total weight of topical composition.

The composition of the exemplary embodiments described herein may be employed in any type of dispenser typically used for gel products, for example pump dispensers. A wide variety of pump dispensers are suitable. Pump dispensers may be affixed to bottles or other free-standing containers. Pump dispensers may be incorporated into wall-mounted dispensers. Pump dispensers may be activated manually by hand or foot pump, or may be automatically activated. Useful dispensers include those available from GOJO Industries under the designations NXT®, TFX™, DPX™, FMX™, ADX™, LTX™, and CXT™ as well as traditional bag-in-box dispensers. Examples of dispensers are described in U.S. Pat. Nos. 5,265,772, 5,944,227, 6,877,642, 7,028,861, 7,611,030, 7,621,426, 8,740,019, 8,991,657, 9,027,790, 9,073,685, 9,101,250, and 9,204,767, all of which are incorporated herein by reference. In one or more embodiments, the dispenser includes an outlet such as a nozzle, through which the composition is dispensed. In some exemplary embodiments, the topical composition is used in dispensers that employ foaming pumps, which combine ambient air or an inert gas and the composition in a mixing chamber and pass the mixture through a mesh screen.

In one or more embodiments, the topical composition is integrated into wipe composition. Wipe compositions in accordance with the exemplary embodiments described herein include at least one alcohol, a C₁₋₁₀ alkanediol enhancer, and are applied to a wipe substrate. In some exemplary embodiments, the wipe composition is alcohol-free.

Wipe substrates used in antimicrobial wipes are further described in U.S. Pat. Nos. 5,686,088, 6,410,499, 6,436,892, 6,495,508, 6,844,308, 9,096,821, which are incorporated herein by reference. In one or more embodiments, the wipe may comprise a laminate formed by spunbonding/meltblowing/spunbonding (SMS). Generally, an SMS material contains a meltblown web sandwiched between two exteriors spunbond webs. SMS materials are further described in U.S. Pat. Nos. 4,041,203, 5,169,706, 5,464,688, and 4,766,029, and are commercially available, for example from Kimberly-Clark Corporation under marks such as Spunguard 7 and Evolution 7. The SMS laminate may be treated or untreated.

In some exemplary embodiments, the topical composition decreases the production and/or activity of pro-inflammatory factors such as interleukins, including interleukin-8 (IL-8). Over-expression of IL-8 is a biomarker of skin irritation. IL-8 is associated with inflammation and plays a role in colorectal cancer. In some exemplary embodiments, a topical composition comprising up to about 15.0 wt. % of the active ingredient in water is able to reduce the relative production and/or activity of pro-inflammatory factors by at least about 50%, or at least about 70%, or at least about 78%, as compared to an otherwise identical control composition without the active ingredient.

In some exemplary embodiments, the topical composition increases the expression of Involucrin. Involucrin is a protein component of human skin and is encoded in humans by the IVL gene. In some exemplary embodiments, a topical composition comprising up to about 15.0 wt. % of an active ingredient is able to increase the relative Involucrin production and/or activity by at least 50%, or at least 70%, or at least 90% or at least 100%, as compared to an otherwise identical control composition not including the active ingredient.

In some exemplary embodiments, the topical composition increases the production and/or activity of cadherins. In some exemplary embodiments, the increased cadherins are desmosomals, such as DCS3. In some exemplary embodiments, a topical composition comprising up to about 15.0 wt. % of an active ingredient is able to increase the relative production and/or activity cadherins, such as DCS3 by at least about 25%, or at least 35%, or at least 50%, or at least 57%, as compared to an otherwise identical control composition not including the active ingredient.

In some exemplary embodiments, a topical composition comprising up to about 15.0 wt. % of an active ingredient increases the production and/or activity of defensins, such as HBD-2. HBD-2 is a low molecular weight AMP produced by epithelia cells and is encoded by the DEFB4 gene. It exhibits potent antimicrobial activity against Gram-negative bacteria and Candida. In some exemplary embodiments, a topical composition comprising up to about 15.0 wt. % of an active ingredient in water is able to increase the relative production and/or activity of defensins, such as HBD-2 by at least about 25%, or at least about 35%, or at least about 45%, or at least about 55%, or at least about 65%, or at least about 75%, or at least about 90%, as compared to an otherwise identical control composition without the active ingredient.

### EXAMPLES

The following examples are included for purposes of illustration and are not intended to limit the scope of the methods described herein.

### Example 1

Topical compositions with Bonicel™ were tested for their ability to decrease production and/or activity of Interleukin 8 (IL-8 or CXCL8) which is a chemokine and proinflammatory cytokine produced by macrophages and other cell types such as epithelial cells. IL-8 is secreted from keratinocytes in skin in response to inflammatory stimuli.

For Control A, human dermal keratinocytes were left untreated. No irritation is expected, and therefore Control A provides a baseline (set as 0). For Control B, IL-8 is induced in human dermal keratinocytes by applying a surfactant mixture that is a combination of sodium laureth sulfate and polyquaternium-10 (set as 100%). For all other samples, the human dermal keratinocytes are co-treated with the surfactant mixture and a composition containing indicated concentration of Bonicel™. Decreased IL-8 production and/or activity reflects an ingredient's anti-irritation activity. In order to carry out the test method, an assay kit was employed that was obtained from R&D Systems: Human CXCL8/IL-8 Duoset ELISA Kit (DY208). ELISA was performed after overnight treatment using by applying 100 μl/well of culture medium according to the manufactory instruction of the ELISA kit. The results were measured using a colorimeter, absorbance was measured at 450 nanometers (nm) within 30 minutes. Wavelength correction was set to 570 nm.

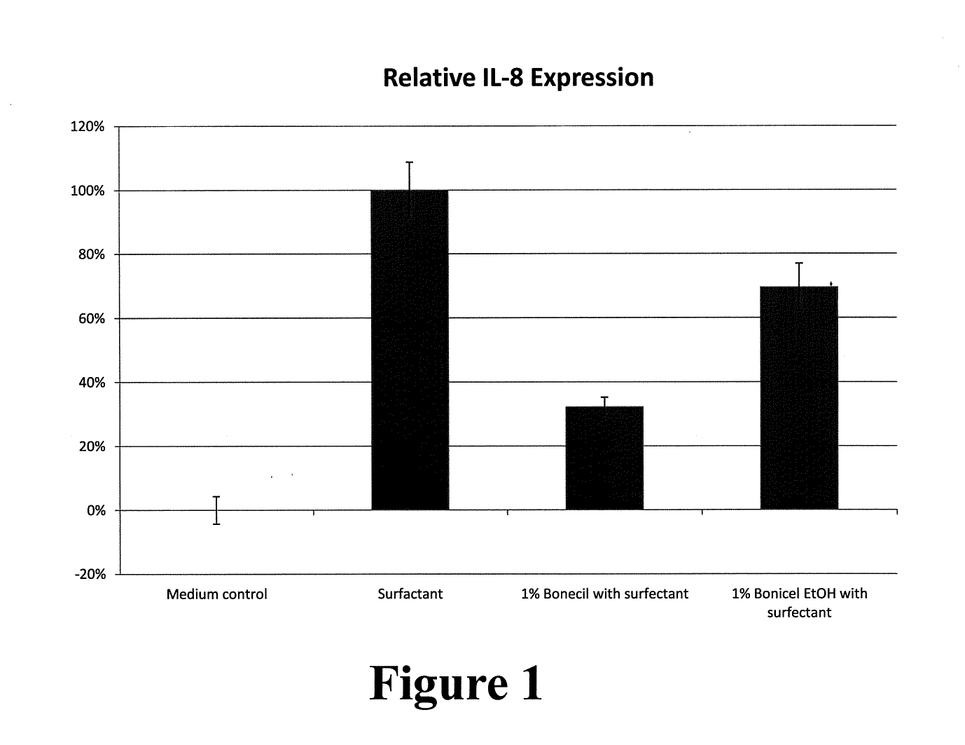

The results showed a topical composition with Bonicel™ was able to reduce the relative IL-8 production and/or activity. A relative decrease in IL-8 production and/or activity of about 78% was observed for a topical composition with 1.0% Bonicel™, water, and a surfactant as compared to a control composition with water and a surfactant. The results are depicted graphically in FIG. 1.

### Example 2

An in vitro study was conducted to study a sample of Bonicel™ specifically for its ability to increase production and/or activity of Involucrin.

Neonatal Human Epidermal Keratinocytes (NHEK; Life Technology, Grand Island, N.Y., USA) were cultured with keratinocyte growth medium (KGM, Medium 154: M-154-500 Life Technology with supplements S-001, Life Technologies). Keratinocytes were treated with the sample compositions in a 6-well plate overnight. After washing with cold phosphate-buffered saline (PBS), total RNAs were prepared from each well. Real-Time Quantitative Reverse Transcription PCR (qRT-PCR) was performed to detect the target genes (Involucrin) expression level using a One-step TaqMan® RT-PCR kit (Life Technologies).

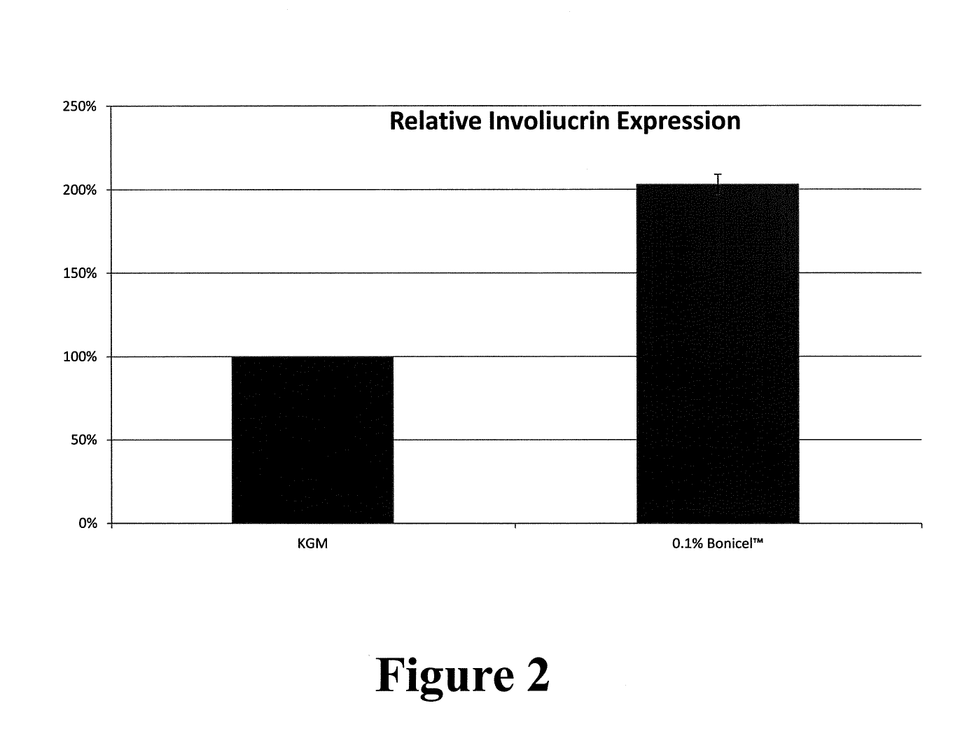

The results showed that Bonicel™ increased the relative production and/or activity of Involucrin. A relative increase in Involucrin production and/or activity of 103% was observed for 0.1% Bonicel™ as compared to the KGM medium control culture. This increase shows that Bonicel™ can stimulate Involucrin production and/or activity in keratinocyte to promote skin keratinocyte differentiations and improve skin barrier function. The results are depicted graphically in FIG. 2.

### Example 3

An in vitro study was conducted to study a sample of Bonicel™ specifically for its ability to increase production and/or activity of desmocollin-3 (DSC3).

Neonatal Human Epidermal Keratinocytes (NHEK; Life Technology, Grand Island, N.Y., USA) were cultured with keratinocyte growth medium (KGM, Medium 154: M-154-500 Life Technology with supplements S-001, Life Technologies). Keratinocytes were treated with the sample compositions in a 6-well plate overnight. After washing with cold phosphate-buffered saline (PBS), total RNAs were prepared from each well. Real-Time Quantitative Reverse Transcription PCR (qRT-PCR) was performed to detect the target genes (DSC3) expression level using a One-step TaqMan® RT-PCR kit (Life Technologies).

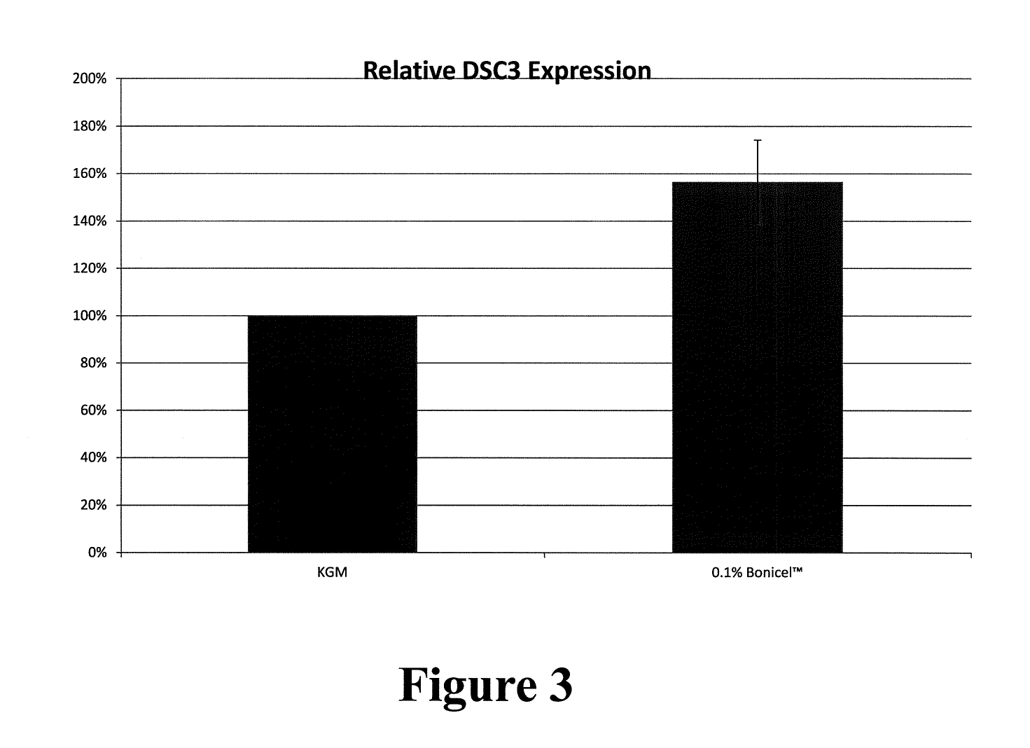

The results showed that Bonicel™ increased the relative production and/or activity of DSC3. A relative increase in DCS3 production and/or activity of about 57% was observed for 0.1% Bonicel™ as compared to the KGM medium culture. This increase shows that Bonicel™ can stimulate skin junction biomarker DSC3 production and/or activity in keratinocytes to improve skin barrier function. The results are depicted graphically in FIG. 3.

### Example 4

In vitro studies were also run with Bonicel™ specifically to determine its ability to simulate production and/or activity of human beta-defensin 2 (HBD-2). Bonicel™ was tested at concentrations of both 0.1% and 1.0% in a water medium.

Neonatal Human Epidermal Keratinocytes (NHEK; Life Technology, Grand Island, N.Y., USA) were cultured with keratinocyte growth medium (KGM, Medium 154: M-154-500 Life Technology with supplements S-001, Life Technologies). NHEK were seeded into 96-well plates at a density of 10000 cells in 200 μl medium per well. After 48 hours, the cells were incubated with varying concentrations of each ingredient solution in a culture medium (KGM) overnight (16 hours) at 37° C., 5% CO₂ and 95% humidity at four replicates for each concentration. Each of these active ingredients was tested at the different concentration of weight percents based on the weight of the total culture. Each of these compositions was compared to a control culture medium.

HBD-2 was detected using HBD-2 ELISA developing kits (commercially available from Peprotech). ELISA were performed according to the manufactory instructions of each kit by adding 100 μl/well of culture medium after overnight treatment. The substrate of ELISA reaction was using the substrate reagent from R&D Systems (DY999), and the reactions were stopped by adding 50 μl of IN H2SO4 in each well. The results were measured using a colorimeter, absorbance was measured at 450 nanometers (nm) within 30 minutes. Wavelength correction was set to 570 nm. The concentration of each sample was calculated using ELISA standard curve.

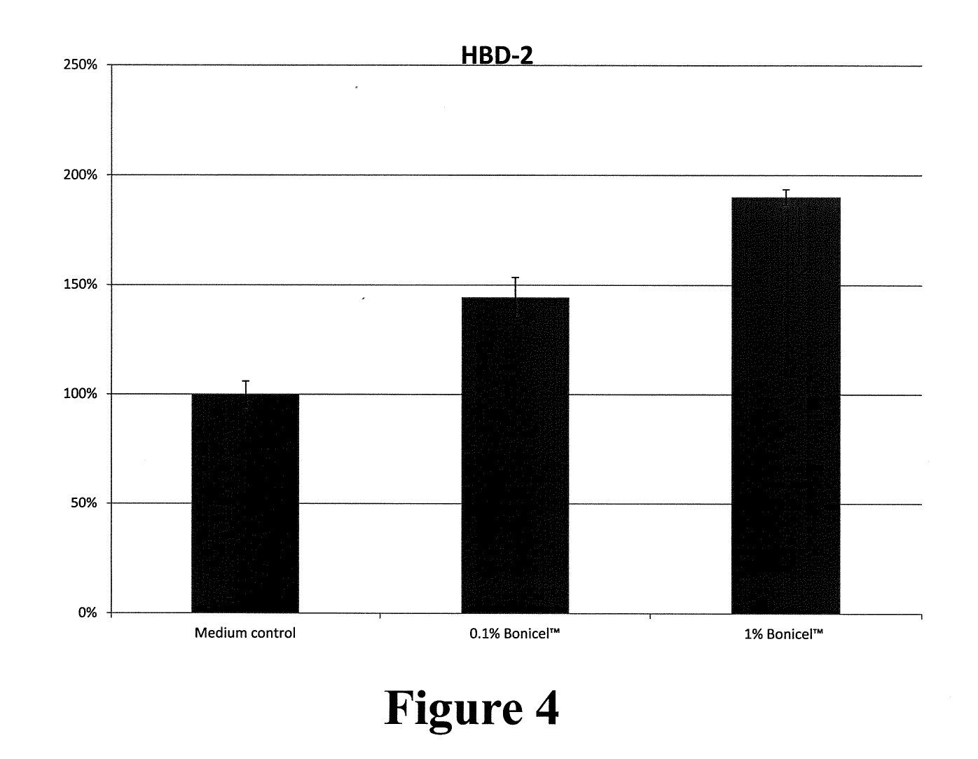

The results showed the Bonicel™ is able to increase the production and/or activity of HBD-2 in a composition with water. Relative increases in HBD-2 production and/or activity of about 44% and about 90% were observed for 0.1% Bonicel™ in a composition with water and 1.0% Bonicel™ in a composition with water, respectively. The results are depicted in FIG. 4.

### Example 5

The effect of exemplary topical compositions was investigated for pathogen blocking potential. Methicillin resistant Staphylococcus aureus strain Mu50 ATCC 33591, Escherichia coli strain K12 was tested against the following exemplary topical compounds: DMEM (cell culture medium, control), 100 nM dexamethasone (DEX, control steroidal anti-inflammatory), 0-5% Ecoskin (α-gluco-oligosaccharide, fructo-oligosaccharide and inactivated Lactobacillus), 0-5% Bacillus ferment, and 0-5% of a prebiotic blend of inulin and fructo-oligosaccahride.

Differentiated colonic epithelial cells were treated with the topical compounds and a bacterial strain was then added individually. The microbe was grown to the mid-log phase in an acceptable medium and the concentration adjusted so that the amount of bacteria added to the wells was approximately 100 microbes per well (in a 96 well tray with total volume of 100 uL). The cells were then incubated with each bacterial strain for one hour. A Gentamicin protection assay was used to determine adhered and invaded bacteria. Polymerase chain reaction (PCR) using 16S gene primers was used to determine the number of adhered bacteria, as well as the number of bacteria that invaded into the host cells.

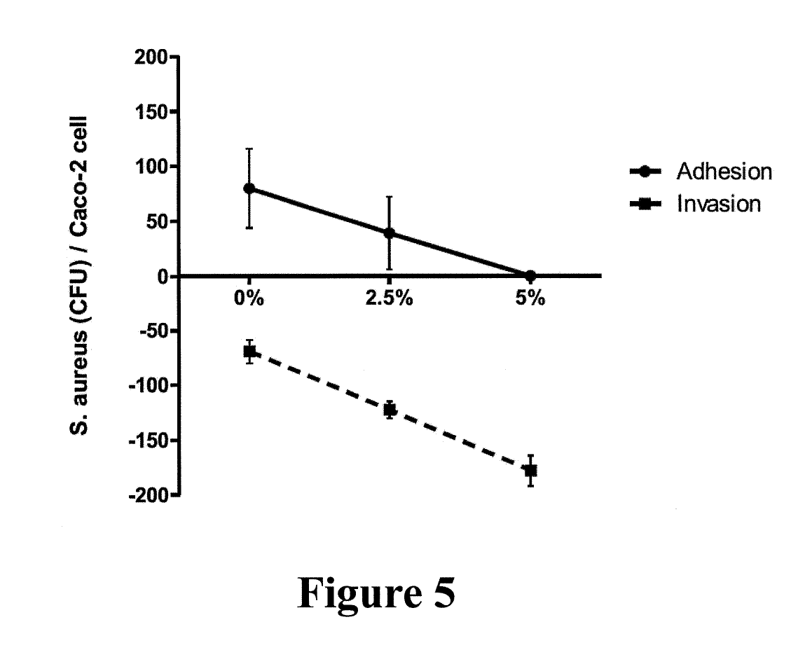

FIG. 5 illustrates the dose-dependent response of Staphylococcus aureus adhesion and invasion potential. Bacillus ferment had a consistent increase in the dose response. Particularly, 5% Bacillus ferment resulted in the lowest adhesion occurrence overall.

Although embodiments of the invention have been described herein, it should be appreciated that many modifications can be made without departing from the spirit and scope of the general inventive concepts. All such modifications are intended to be included within the scope of the invention.

1. A topical cleansing composition for restoring skin's natural balance of bacteria, the topical cleansing composition comprising;
about 0.005 wt. % to about 15.0 wt. % of an active ingredient; and
at least one primary and at least one secondary surfactant;
wherein the active ingredient comprises one or more of a probiotic, probiotic derivative, and a prebiotic and wherein the topical cleansing composition increases the production and/or activity of defensins by at least about 44%, relative to an otherwise identical topical composition without the active ingredient.

2. The topical cleansing composition of claim 1, wherein the primary surfactant is sodium laureth sulfate.

3. The topical cleansing composition of claim 1, wherein the secondary surfactant is selected from one or more of cocamidopropyl betaine, disodium cocoamphodiacetate, cocamidopropyl hydroxysultaine, and lauryl glucoside.

4. The topical cleansing composition of claim 1, wherein the active ingredient is a probiotic derived ingredient.

5. The topical cleansing composition of claim 1, wherein the probiotic or probiotic derived ingredient is selected from a strain of one or more of Lactobacillus, Clostridia, Bifidobacterium, Saccharomyces, Lactococcus, Pedicoccus, Enterococcus, Escherichia, Alcaligenes, Corynebacterium, Bacillus, and Propionibacterium.

6. The topical cleansing composition of claim 1, wherein the probiotic or probiotic derived ingredient is Bacillus ferment.

7. The topical cleansing composition of claim 1, wherein the topical cleansing composition comprises from about 0.05 to about 5.0 wt. % active ingredient, based on the weight of the total topical cleansing composition.

8. The topical cleansing composition of claim 1, wherein the topical cleansing composition comprises from about 0.1 to about 1.0 wt. % active ingredient, based on the weight of the total topical cleansing composition.

9. The topical cleansing composition of claim 1, wherein the topical composition further comprises one or more skin conditioning agents.

10. The topical cleansing composition of claim 9, wherein the one or more skin conditioning agents comprises one or more humectants, selected from the group consisting of propylene glycol, hexylene glycol, 1,4-dihydroxyhexane, 1,2,6-hexanetriol, sorbitol, butylene glycol, caprylyl glycol, propanediols, such as methyl propane diol, dipropylene glycol, triethylene glycol, glycerin (glycerol), polyethylene glycols, ethoxydiglycol, polyethylene sorbitol, glyceryl caprylate/caprate and combinations thereof.

11. The topical cleansing composition of claim 10, wherein the humectant comprises glycerin.

12. (canceled)

13. The topical cleansing composition of claim 1, wherein the topical sanitizing composition comprises one or more plug preventing additives.

14. The topical cleansing composition of claim 13, wherein the plug-preventing additive is present in an amount up to about 20.0 wt. %, based on the weight of the total topical cleansing composition.

15. The topical cleansing composition of claim 1, wherein the topical composition further comprises one or more moisturizing esters, selected from the group consisting of cetyl myristate, cetyl myristoleate, and other cetyl esters, diisopropyl sebacate, isopropyl myristate, and combinations thereof.

16. The topical cleansing composition of claim 13, wherein the moisturizing ester is present in an amount up to about 10.0 wt. %, based on the weight of the total topical cleansing composition.

17. (canceled)

18. (canceled)

19. A method of skin treatment for reducing irritation on the skin, the method comprising:
applying a topical cleansing composition to a skin surface, wherein the topical cleansing composition comprises:
about 0.005 wt. % to about 15.0 wt. % of an active ingredient; and
at least one primary and at least one secondary surfactant; and
rinsing the topical cleansing composition off with water,
wherein the active ingredient comprises one or more of a probiotic, a probiotic derivative, and a prebiotic and wherein the topical cleansing composition reduces the production and/or activity of pro-inflammatory markers by a statistically significant amount, as compared to an otherwise identical composition without the active ingredient.

20. The method of claim 19, wherein the topical cleansing composition decreases the production and/or activity of pro-inflammatory markers by at least about 78%, relative to an otherwise identical topical composition without the active ingredient.

21. A topical cleansing composition for increasing the production and/or activity of antimicrobial peptides, the topical cleansing composition comprising;
about 0.005 wt. % to about 15.0 wt. % of an active ingredient; and
at least one primary and at least one secondary surfactant;
wherein the active ingredient comprises one or more of a probiotic, a probiotic derivative, and a prebiotic and wherein the topical cleansing composition increases the production and/or activity of at least one antimicrobial peptide by a statistically significant amount, as compared to an otherwise identical topical composition without the active ingredient.

22. The topical cleansing composition of claim 21, wherein the topical cleansing composition increases the production and/or activity of defensins by at least about 44%, relative to an otherwise identical topical composition without the active ingredient.

23. The topical cleansing composition of claim 21, wherein the topical cleansing composition increases the production and/or activity of cadherins by at least about 57%, relative to an otherwise identical topical composition without the active ingredient.
